(19) United States
(12) Patent Application Publication
(10) Pub. No.: US 2024/0387014 A1
(43) Pub. Date: Nov. 21, 2024

(54) DOMAIN-ADAPTIVE PRE-TRAINING OF INSTRUCTION-TUNED LLMS FOR RADIOLOGY REPORT IMPRESSION GENERATION

(71) Applicant: Siemens Healthineers AG, Forchheim (DE)

(72) Inventors: Sanjeev Kumar Karn, Plainsboro, NJ (US); Rikhiya Ghosh, Livingston, NJ (US); Kusuma P, Rayasandra, Bengaluru (IN); Oladimeji Farri, Upper Saddle River, NJ (US)

(21) Appl. No.: 18/602,071

(22) Filed: Mar. 12, 2024

(30) Foreign Application Priority Data
May 18, 2023 (IN) 202311034814

(51) Int. Cl. G16H 15/00; G16H 50/70

(52) U.S. Cl. CPC G16H 15/00; G16H 50/70

(57) ABSTRACT

Systems and methods for performing a clinical task using a trained language model are provided. Input medical data associated with a medical domain is received. A clinical task is performed based on the input medical data using a trained language model. Results of the clinical task are output. The trained language model is trained by: receiving domain-specific training data associated with the medical domain and training a pretrained, instruction-tuned language model for the medical domain using the domain-specific training data.

### CROSS-REFERENCE TO RELATED APPLICATIONS

This application is based upon and claims the benefit of priority from Indian patent application Ser. No. 20/231,1034814, filed May 18, 2023, the disclosure of which is incorporated herein by reference in its entirety.

### TECHNICAL FIELD

The present invention relates generally to domain-adaptive pre-training of instruction-tuned LLMs (large language models), and in particular to domain-adaptive pre-training of instruction-tuned LLMs for radiology report impression generation.

### BACKGROUND

Radiology reports are the primary method for radiologists to communicate their interpretation of medical images to the ordering physicians. Such radiology reports typically include a findings section and an impressions section, among other sections. The findings section describes abnormalities and diagnoses, while the impressions section summarizes the findings and highlights major abnormalities and recommendations.

Recently, pretrained language models have been proposed for automatically generating the impressions section of a radiology report from the findings section of the radiology report. Pretrained language models are typically trained on vast, diverse training datasets, allowing them to capture various linguistic patterns. In one conventional approach, pretrained language models are trained via the pretrain-and-finetune approach for learning downstream tasks (such as, e.g., impressions generation) with significant training data scarcity. In another conventional approach, the pretrain-and-prompt-tune approach (commonly referred to as prompt-tuning) has been proposed for training pretrained language models. Instead of fine-tuning the pretrained language models as in the pretrain-and-finetune approach, the objectives of downstream tasks are reconstructed using textual prompts in the pretrain-and-prompt-tune approach. Multitask prompted finetuning (also known as instruction tuning) is a type of large-scale pretrain-and-prompt-tune, where finetuning of large pretrained language models is performed with training datasets representing various natural language processing tasks defined by instructions as natural language prompts. However, pretrained language models trained according to such conventional approaches are limited in their understanding of radiology reports and tend to generate either verbose or incomplete impressions sections, primarily due to insufficient exposure to medical text data during training.

### BRIEF SUMMARY OF THE INVENTION

In accordance with one or more embodiments, systems and methods for performing a clinical task using a trained language model are provided. Input medical data associated with a medical domain is received. A clinical task is performed based on the input medical data using a trained language model. Results of the clinical task are output. The trained language model is trained by receiving domain-specific training data associated with the medical domain and training a pretrained, instruction-tuned language model for the medical domain using the domain-specific training data.

In one embodiment, the pretrained, instruction-tuned language model is trained by: performing general pretraining of a language model using non-domain-specific training data and performing instruction tuning on the general pretrained language model using labeled training data. In one embodiment, a same loss function is used for performing the general pretraining, performing the instruction tuning, and the training.

In one embodiment, the pretrained, instruction-tuned language model is trained by updating only parameters of certain layers of the pretrained, instruction-tuned language model at each iteration.

In one embodiment, the pretrained, instruction-tuned language model is trained by adding domain-specific vocabulary for the medical domain to the pretrained, instruction-tuned language model.

In one embodiment, the input medical data comprises a findings section of a radiology report and the clinical task comprises generation of an impressions section of the radiology report.

In one embodiment, the medical domain is radiology. In one embodiment, the trained language model is a trained large language model.

In accordance with one or more embodiments, systems and methods for training a language model for a medical domain are provided. Domain-specific training data associated with a medical domain is received. A pretrained, instruction-tuned language model is trained for the medical domain using the domain-specific training data. The trained language model is output.

These and other advantages of the invention will be apparent to those of ordinary skill in the art by reference to the following detailed description and the accompanying drawings.

### BRIEF DESCRIPTION OF THE DRAWINGS

FIG. 1 shows a method for training a language model for a medical domain, in accordance with one or more embodiments;

FIG. 2 shows a method for performing a clinical task using a trained language model, in accordance with one or more embodiments;

FIG. 3 shows a table comparing performance of a system trained in accordance with one or more embodiments with conventionally trained system;

FIG. 4 shows a table of results of various domain adaptation approaches;

FIG. 5 shows an exemplary artificial neural network that may be used to implement one or more embodiments;

FIG. 6 shows a convolutional neural network that may be used to implement one or more embodiments;

FIG. 7 shows a schematic structure of a recurrent machine learning model that may be used to implement one or more embodiments; and

FIG. 8 shows a high-level block diagram of a computer that may be used to implement one or more embodiments.

### DETAILED DESCRIPTION

The present invention generally relates to methods and systems for domain-adaptive pre-training of instruction-tuned LLMs for radiology report impression generation. Embodiments of the present invention are described herein to give a visual understanding of such methods and systems. A digital image is often composed of digital representations of one or more objects (or shapes). The digital representation of an object is often described herein in terms of identifying and manipulating the objects. Such manipulations are virtual manipulations accomplished in the memory or other circuitry/hardware of a computer system. Accordingly, is to be understood that embodiments of the present invention may be performed within a computer system using data stored within the computer system. Further, reference herein to pixels of an image may refer equally to voxels of an image and vice versa.

Embodiments described herein provide for a three-stage approach for training a pretrained language model for performing domain-specific tasks: 1) general pretraining, 2) prompt-tuning, and 3) domain-specialized pretraining. Stages 1 and 2 (i.e., general pretraining and prompt-tuning) correspond to the stages in the pretrain-and-finetune approach. Embodiments described herein thus provide for an extension to the pretrain-and-finetune approach by adding the domain-specialized pretraining stage for training pretrained language models for a particular medical domain, thereby resulting a general-pretrain-prompt-tune-and-special-pretrain approach. Advantageously, language models trained in accordance with embodiments described herein have been experimentally found to significantly improve performance of the impressions generation task, which will simplify and improve adaptation by clinicians.

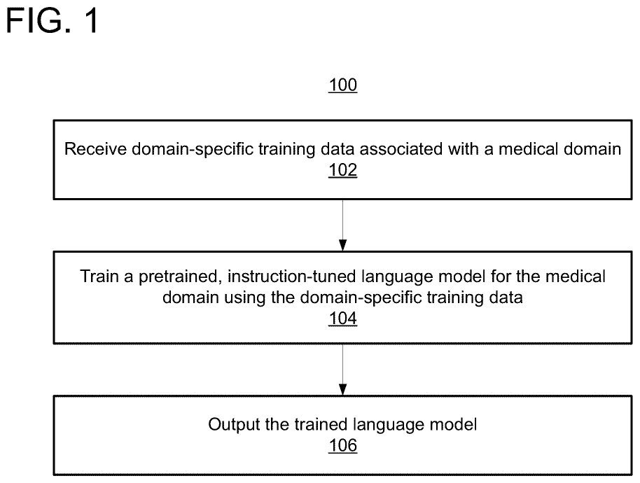

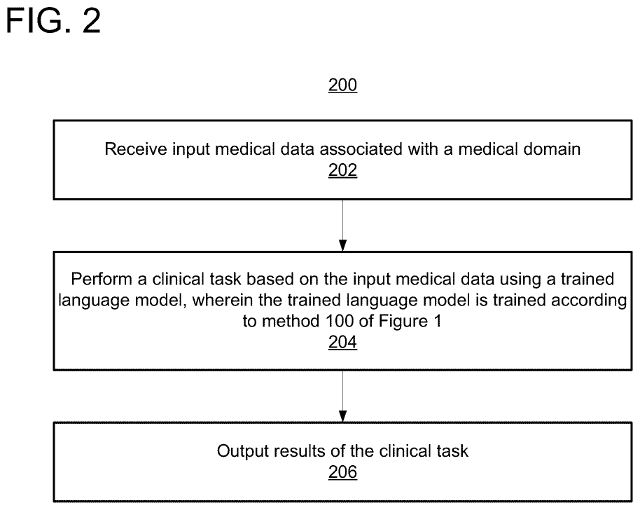

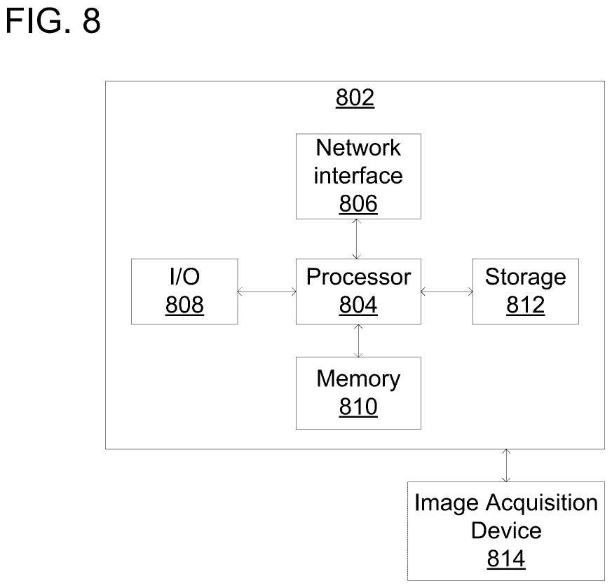

FIG. 1 shows a method 100 for training a language model for a medical domain, in accordance with one or more embodiments. The steps of method 100 may be performed by one or more suitable computing devices, such as, e.g., computer 802 of FIG. 8. The steps of method 100 are performed during an offline or training stage for training the language model. Once trained, the trained language model may be applied during an online or inference stage, e.g., to perform the steps of method 200 of FIG. 2.

At step 102 of FIG. 1, domain-specific training data associated with a medical domain is received. In one embodiment, the medical domain is radiology. However, the medical domain may any other suitable medical domain.

In one embodiment, the domain-specific training data comprises text-based training data. For example, the text-based training data may comprise radiology reports of a patient (comprising a findings section and an impression section). However, the text-based training data may comprise any other suitable text-based data of the patient, such as, e.g., other types of reports (e.g., clinical reports), demographic information, vital signs, medical history, family history, laboratory results, measurements and information extracted from medical images, etc. of the patient.

In one embodiment, the domain-specific training data may alternatively or additionally comprise non-text-based training data. For example, the non-text-based training data may comprise training images. The training images may be associated with the text-based training data and depict one or more anatomical objects, such as, e.g., organs, bones, vessels, tumors or other abnormalities, or any other anatomical objects of interest of the patient. The training images may be of any suitable modality, such as, e.g., CT (computed tomography), MRI (magnetic resonance imaging), US (ultrasound), x-ray, or any other medical imaging modality or combinations of medical imaging modalities. The training images may comprise 2D (two dimensional) images and/or 3D (three dimensional) volumes.

The domain-specific training data may be received, for example, by directly receiving the domain-specific training data from an image acquisition device (e.g., image acquisition device 814 of FIG. 8) as the training images are acquired, by loading the one or more domain-specific training data from a storage or memory of a computer system (e.g., memory 810 or storage 812 of computer 802 of FIG. 8), or by receiving the domain-specific training data from a remote computer system (e.g., computer 802 of FIG. 8). Such a computer system or remote computer system may comprise one or more patient databases, such as, e.g., an EHR (electronic health record), EMR (electronic medical record), PHR (personal health record), HIS (health information system), RIS (radiology information system), PACS (picture archiving and communication system), LIMS (laboratory information management system), or any other suitable database or system.

At step 104 of FIG. 1, a pretrained, instruction-tuned language model is trained for the medical domain using the domain-specific training data. The language model is an AI/ML based model trained for predicting linguistic sequences. In one embodiment, the language model is an LLM (large language model). However, the language model may be any other suitable language model. For example, the language model may be a small language model, which uses a relatively smaller neural network, has fewer parameters, and is trained on less training data as compared with an LLM.

The LLM may be any suitable pretrained deep learning based LLM. For example, the LLM may be based on the transformer architecture, which uses an attention mechanism to capture long-range dependencies in text. One example of a transformer-based architecture is GPT (generative pre-training transformer), which has a multilayer transformer decoder architecture that may be pretrained to optimize the next token prediction task and then fine-tuned with labelled data for various downstream tasks. Other exemplary transformer-based architectures include BLOOM (BigScience Large Open-science Open-access Multilingual Language Model) and BERT (Bidirectional Encoder Representations from Transformers). In some embodiments, the LLM may be a multi-modal LLM to receive, e.g., the training images in addition to the text-based training data.

The language model is pretrained during a prior stage or stages to form the pretrained, instruction-tuned language model. In one embodiment, a general pretraining stage is first performed for pretraining the language model using a large and diverse corpus of general (i.e., non-domain-specific) training data to learn the intricacies of language patterns, semantics, and syntax. The pretrained language model is then fine-tuned during an instruction tuning stage using training datasets representing various natural language processing tasks, defined by instructions as natural language prompts.

During the general pretraining stage, the language model receives as input training text data, tokenizes the training text data into smaller units (e.g., words or sub-words), and converts each token into a high-dimensional vector representation, referred to as word embeddings. The word embeddings capture the semantic and syntactic information of the text in the corpus. The tokenized and embedded text data is then used to train the language model. During training, the language model generates as output a predicted next token in the sequence given the previous tokens. The language model's parameters are updated iteratively through optimization algorithm to minimize the prediction error. The general pretraining stage aims to expose the language model to a wide range of linguistic patterns and contexts, enabling it to develop a rich understanding of language structure and semantics.

The language model may be trained during the general pretraining stage in an unsupervised manner, a self-supervised manner, or any other suitable approach. In unsupervised learning, the language model learns to represent the underlying structure of the domain-specific training data without explicit supervision. In this manner, the language model is trained to predict the next token in a sequence given the previous tokens. Any suitable loss function may be used for unsupervised learning, such as, e.g., cross-entropy loss, contrastive loss, triplet loss, mean squared error loss, etc. In self-supervised learning, training signals are generated from the input domain-specific training data itself. Self-supervised learning involves creating surrogate tasks from the input domain-specific training data that can be used to train the model. In one example, the self-supervised task is masked language modeling, where certain words in a sentence are masked and the language model is trained to predict the masked words based on the context provided by the surrounding words. Any suitable loss function may be used for self-supervised learning, such as, e.g., cross-entropy loss, masked language modeling loss, triplet loss, etc.

During the instruction tuning stage, the pretrained language model is fine-tuned on various downstream natural language processing tasks to help the language model adapt its learned representations to the natural language processing tasks. Exemplary natural language processing tasks include text classification, named entity recognition, machine translation, sentiment analysis, question answering, or any other natural language processing task. Instruction tuning is performed on labeled training text data corresponding to the tasks. During instruction tuning, the pretrained language model receives as input the training text data and generates as output results for the tasks. The output results are compared with the labels and the parameters of the pretrained language model are updated via backpropagation to minimize a task-specific loss function (e.g., cross-entropy loss, means squared error loss, dice loss, etc.) over a number of iterations. A number (e.g., 300) of the natural language processing tasks may be performed in parallel during instruction tuning. Once fine-tuned via instruction tuning, the pretrained, instruction-tuned language model is provided.

The pretrained, instruction-tuned language model is then further trained for the medical domain at step 104 during a domain-specialized pretraining stage using the domain-specific training data. The domain-specialized pretraining stage may be performed similarly to the general pretraining stage but using the domain-specific training data. In one embodiment, the same loss function is utilized in each of the three training stages (i.e., general pretraining, instruction tuning, and domain-specialized pretraining).

In one embodiment, the domain-specialized pretraining is performed in a parameter efficient manner. During the domain-specialized pretraining, there is a chance of catastrophic forgetting of what was learned during the general pretraining stage and the instruction tuning stage. To avoid catastrophic forgetting, the domain-specialized pretraining is performed by either only updating parameters of certain layers of the language model or other parameter efficient fine-tuning techniques at each iteration or epoch.

In one embodiment, the domain-specialized pretraining stage may be performed with vocabulary expansion to increase the size of the vocabulary that the language model can understand and generate. Vocabulary expansion may be performed by, for example, using adding domain-specific vocabulary for the medical domain to the language model to better capture language patterns for the medical domain. Vocabulary expansion may be performed using any other suitable approach, such as, e.g., word segmentation, sub-word tokenization, dynamic vocabulary, etc. By expanding vocabulary, embedding layer parameters of the model are expanded. During training, additional loss is used to only update new embedding parameters. Vocabulary expansion is further described in Ghosh et al., “RadLing: Towards efficient radiology report understanding”, Proceedings of the 61st Annual Meeting of the Association for Computational Linguistics, Volume 5: Industry Track, pages 640-651, 2023, the disclosure of which is incorporated by reference herein in its entirety.

In one embodiment, optionally, the pretrained, instruction-tuned language model may be fine-tuned for performing a particular clinical task (e.g., impressions generation).

At step 106 of FIG. 1, the trained language model is output. For example, the trained language model can be output by storing the trained language model on a memory or storage of a computer system (e.g., memory 810 or storage 812 of computer 802 of FIG. 8) or by transmitting the trained language model to a remote computer system (e.g., computer 802 of FIG. 8).

FIG. 2 shows a method 200 for performing a clinical task using a trained language model, in accordance with one or more embodiments. The steps of method 200 may be performed by one or more suitable computing devices, such as, e.g., computer 802 of FIG. 8. The steps of method z00 are performed during an online or inference stage to perform the clinical task using a trained language model. The trained language model is trained during a prior offline or training stage, e.g., according to method 100 of FIG. 1.

At step 202 of FIG. 2, input medical data associated with a medical domain is received. In one embodiment, the medical domain is radiology. However, the medical domain may any other suitable medical domain.

In one embodiment, the input medical data comprises text-based medical data. For example, the text-based medical data may comprise a findings section of a radiology report. However, the text-based medical data may comprise any other suitable text-based data of a patient, such as, e.g., other types or sections of reports (e.g., clinical reports), demographic information, vital signs, medical history, family history, laboratory results, measurements and information extracted from medical images, etc. of the patient.

In one embodiment, the input medical data may alternatively or additionally comprise non-text-based medical data. For example, the non-text-based medical data may comprise medical images of the patient. The medical images may be associated with the text-based medical data and depict one or more anatomical objects. The medical images may be of any suitable modality, such as, e.g., CT, MRI, US, x-ray, or any other medical imaging modality or combinations of medical imaging modalities. The medical images may comprise 2D images and/or 3D volumes.

The input medical data may be received, for example, by directly receiving the input medical data from an image acquisition device (e.g., image acquisition device 814 of FIG. 8) as the medical images are acquired, by loading the input medical data from a storage or memory of a computer system (e.g., memory 810 or storage 812 of computer 802 of FIG. 8), or by receiving the input medical data from a remote computer system (e.g., computer 802 of FIG. 8). Such a computer system or remote computer system may comprise one or more patient databases.

At step 204 of FIG. 2, a clinical task is performed based on the input medical image using a trained language model. The trained language model is trained according to method 100 of FIG. 1 during a prior offline or training stage. In one embodiment, the clinical task is impressions generation to generate an impressions section of a radiology report from a findings section of the radiology report. However, the clinical task may comprise any other suitable clinical task, such as, e.g., medical question-answering, visual question-answering, information extraction, image captioning, etc.

The language model is an AI/ML based model trained for predicting linguistic sequences. In one embodiment, the language model is an LLM. The LLM may be any suitable pretrained deep learning based LLM. However, the language model may be any other suitable language model (e.g., small language model). The language model receives as input one or more prompts comprising the input medical data and generates as output text-based results of the clinical task. A prompt refers to input to a language model for generating a response. The prompt may be received, for example, from a computer system via one or more APIs (application programming interfaces) or from a user interacting with a computer system.

At step 206 of FIG. 1, results of the clinical task are output. For example, the results of the clinical task can be output by displaying the results of the clinical task on a display device (I/O 808 of computer 802), by storing the results of the clinical task on a memory or storage of a computer system (e.g., memory 810 or storage 812 of computer 802 of FIG. 8), or by transmitting the results of the clinical task to a remote computer system (e.g., computer 802 of FIG. 8).

Embodiments described herein were experimentally validated. Domain-adaptive pretraining was performed using the MIMIC (Medical Information Mart for Intensive Care)-IV dataset, which contains over 2.3 million radiology reports from 237,000 patients and amounts to approximately 616 million tokens using the Bloomz tokenizer. After preprocessing, only 1.4 million reports were utilized with 190 million tokens.

Finetuning for impression generations was performed utilizing three datasets: MIMIC-III, MIMIC-CSR, and cheXpert, presplit into findings and impressions sections. For MIMIC-III, there were 59,320 reports in the training dataset, 7,413 in the validation dataset, 6,526 in the test dataset, and 6,531 in the hidden test dataset. Most reports (91.4%) pertain to CT imaging, with the most represented anatomy being the head (52.8%). Although the task related to MIMIC-CXR/cheXpert datasets is multimodal, only radiology reports were used for fine-tuning and inference. The MIMIC-CXR training dataset had 125,417 reports for training, 991 for validation, and 1,624 for testing. The hidden dataset, a cheXpert dataset, contains 1,000 reports for evaluation.

The methods comprise preprocessing, domain-adaptive pretraining, fine-tuning, and inference.

In the preprocessing step, Regex-based cleaning and normalization were used to remove irrelevant characters and texts from the report. Special tokens for de-identified text were incorporated and distinct sections, such as findings and impressions sections, were identified. Reports with both findings and impressions sections and fewer than 512 tokens were selected.

For domain adaptive pretraining, a version of GPT-powered Bloom was used. Bloom has multiple versions based on parameters. The largest has 176 billion parameters, 70 layers, 112 attention heads, and 14,336-dimensional hidden layers. Bloomz, a massive multitask instruction-tuned version of Bloom, was used, specifically its Bloombz-7b1 variant with 7 billion parameters, 30 layers, and 4,096-dimensional hidden layers for domain adaption.

Following the general-pretrain-prompt-tun-and-special-pretrain approach in accordance with embodiments described herein, Bloombz-7b1 was continuously pretrained using cross-entropy loss on auto-regressively generated tokens from the findings and impressions sections of MIMIC-IV reports.

The domain-specific task for fine-tuning an LLM was radiology report summarization. Using standard prompt-based fine-tuning, findings and TL;DR was employed as prompts, and Bloomz-7b1 was fine-tuned by comparing auto-regressively generated summary tokens to ground-truth impressions using cross-entropy loss. This method ensures fine-tuning consistency with base Bloom and intermediate Bloomz's pretraining and instruction-tuning objectives. To prevent catastrophic forgetting, Bloombz-7b1's trainable parameters are minimized by only allowing the last layer to be modified.

The inference pipeline utilized the trained model to generate impressions based on the given findings. Evaluation metrics for the generated results include Rouge scores, F1RadGraph, Bertscore, and F1CheXbert for the MIMIC0CSR and cheXpert datasets.

Two experimental runs were proposed for the summarization task. 1) Radiology Domain Adaptive Pretraining (RadBloomz) with MIMIC-IV and zero-shot inference. The Bloombz-7b1 model was fine-tuned with a casual language objective on MIMIC-IV radiology reports, creating RadBloomz. With a sequence length of 512, training batch size of 64, validation batch size of 32, learning rate of 3e−5, and AdamW optimizer, the best zero-shot inference is achieved at 24 k steps. 2) RadBloomz finetuned with MIMIC-III. Following the pretrain-and-finetune approach, RadBloomz is further fine-tuned with the MIMIC-III dataset for radiology report summarization. Using the same hyperparameters and training configuration, the best results are achieved at 2697 steps.

All experiments were conducted on the same infrastructure, utilizing eight Tesla A100 SXM4 GPUs (80 GB memory each) and Deepspeed zero-3 configuration with BF16 enabled. A sampling-based technique was used to generate summarizes for the model output distribution, with a maximum of 128 tokens, top_k set to 50, and top_k at 0.7.

RadBloomz was evaluated against other systems using ROUGE for n-gram overall and F1RadGraph for fact overall.

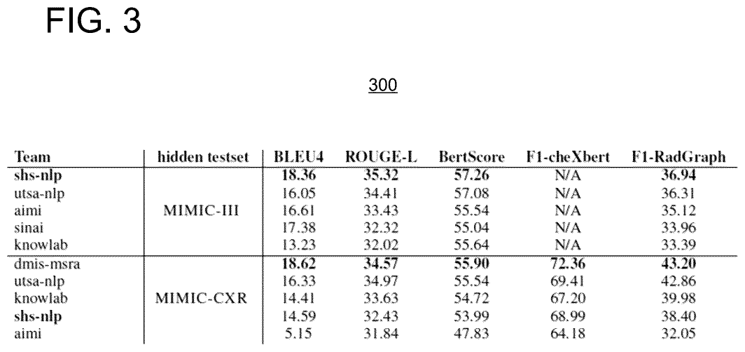

FIG. 3 shows a table 300 presenting the performance of the top five system on the two categories of hidden test data for radiology report summarization task. Table 300 shows RadBloomz's (shs-nlp team) performance on MIMIC-CSR and MIMIC-III hidden test datasets. The MIMIC-III hidden dataset contains only reports, while MIMIC-CXR includes reports and images. Since the system is text-based, MIMIC-III is a more suitable evaluation. RadBloomz ranks first in MIMIC-III and fourth in MIMIC-CXR among all submitted systems, demonstrating the effectiveness of the domain adaptation technique in a multi-modal task.

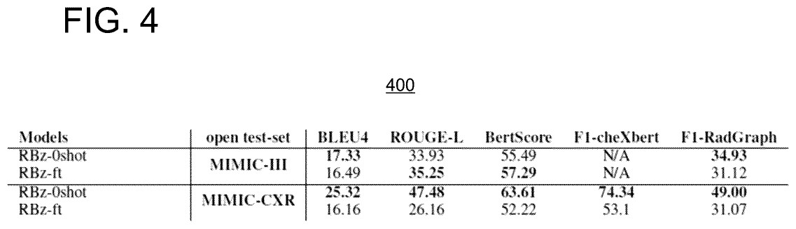

FIG. 4 shows a table 400 of results for various domain adaptation approaches on different test splits of the radiology report summarization task. In table 400, the standard pretrain-and-finetune approach is compared with the pretrain-prompt-tune-pretrain-and-zero-shot approach in accordance with embodiments described herein on the radiology report summarization test data. It is observed that while finetuning with MIMIC-III improves Rouge-L and Bertscore metrics for the MIMIC-III test dataset, Bertscore, F1-RadGraph, and F1-cheXbert scores are lower for the finetuned model. This indicates that the domain adaptation paradigm is sufficient for achieving higher performance without requiring task-specific finetuning.

A thorough error analysis on the open test datasets revealed that many generated impressions receive low scores for both Rouge and F1-RadGraph when the ground-truth radiology report impressions does not mention any abnormalities. For instance, the generated impression “normal MRI of the cervical spine” and the ground truth impression “negative study” are semantically similar. However, these n-gram overlap-based scores fail to recognize their semantic relatedness.

Similarly, it was observed that similar findings sometimes generate different impressions. For example, impressions can be as detailed as: “near complete opacification of the ethmoid air cells and sphenoid sinuses, moderate air-fluid level with mucosal thickening of the right maxillary sinus, and moderate mucosal thickening of the left maxillary sinus.” Meanwhile, similar findings in another report might be summarized as “pansinusitis, as described above.”

Using the domain-specific task of radiology report summarization, it was demonstrated that the general-pretrain-prompt-tune-and-special-pretrain trained LLM model outperforms the standard pretrain-and-finetune approach, even in a zero-shot setting. The system ranked first among participating systems in the hidden-test category.

Embodiments described herein are described with respect to the claimed systems as well as with respect to the claimed methods. Features, advantages or alternative embodiments herein can be assigned to the other claimed objects and vice versa. In other words, claims and embodiments for the systems can be improved with features described or claimed in the context of the respective methods. In this case, the functional features of the method are implemented by physical units of the system.

Furthermore, certain embodiments described herein are described with respect to methods and systems utilizing trained machine learning models, as well as with respect to methods and systems for providing trained machine learning models. Features, advantages or alternative embodiments herein can be assigned to the other claimed objects and vice versa. In other words, claims and embodiments for providing trained machine learning models can be improved with features described or claimed in the context of utilizing trained machine learning models, and vice versa. In particular, datasets used in the methods and systems for utilizing trained machine learning models can have the same properties and features as the corresponding datasets used in the methods and systems for providing trained machine learning models, and the trained machine learning models provided by the respective methods and systems can be used in the methods and systems for utilizing the trained machine learning models.

In general, a trained machine learning model mimics cognitive functions that humans associate with other human minds. In particular, by training based on training data the machine learning model is able to adapt to new circumstances and to detect and extrapolate patterns. Another term for “trained machine learning model” is “trained function.”

In general, parameters of a machine learning model can be adapted by means of training. In particular, supervised training, semi-supervised training, unsupervised training, reinforcement learning and/or active learning can be used. Furthermore, representation learning (an alternative term is “feature learning”) can be used. In particular, the parameters of the machine learning models can be adapted iteratively by several steps of training. In particular, within the training a certain cost function can be minimized. In particular, within the training of a neural network the backpropagation algorithm can be used.

In particular, a machine learning model, such as, e.g., the pretrained, instruction-tuned language model utilized at step 104 of FIG. 1 and the trained language model utilized at step 204 of FIG. 2, can comprise, for example, a neural network, a support vector machine, a decision tree and/or a Bayesian network, and/or the machine learning model can be based on, for example, k-means clustering, Q-learning, genetic algorithms and/or association rules. In particular, a neural network can be, e.g., a deep neural network, a convolutional neural network or a convolutional deep neural network. Furthermore, a neural network can be, e.g., an adversarial network, a deep adversarial network and/or a generative adversarial network.

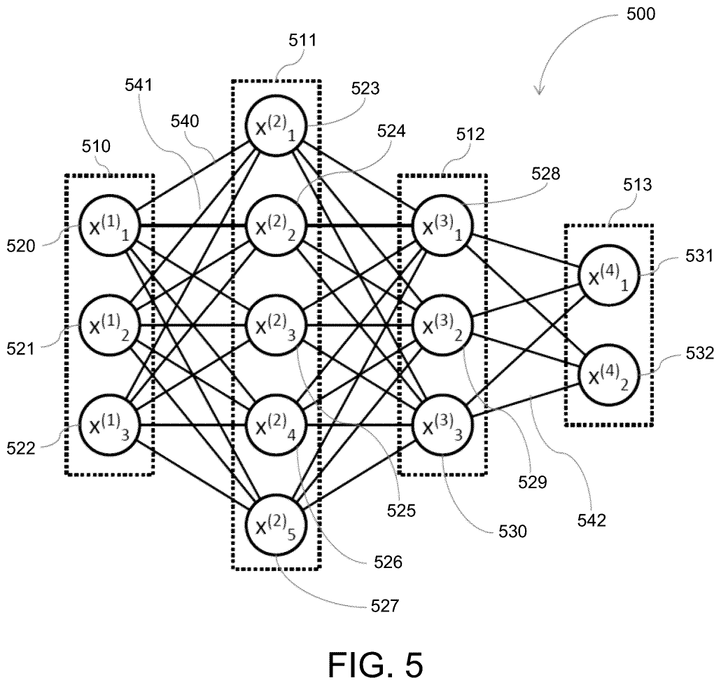

FIG. 5 shows an embodiment of an artificial neural network 500 that may be used to implement one or more machine learning models described herein. Alternative terms for “artificial neural network” are “neural network”, “artificial neural net” or “neural net”.

The artificial neural network 500 comprises nodes 520, . . . , 532 and edges 540, . . . , 542, wherein each edge 540, . . . , 542 is a directed connection from a first node 520, . . . , 532 to a second node 520, . . . , 532. In general, the first node 520, . . . , 532 and the second node 520, . . . , 532 are different nodes 520, . . . , 532, it is also possible that the first node 520, . . . , 532 and the second node 520, . . . , 532 are identical. For example, in FIG. 5 the edge 540 is a directed connection from the node 520 to the node 523, and the edge 542 is a directed connection from the node 530 to the node 532. An edge 540, . . . , 542 from a first node 520, . . . , 532 to a second node 520, . . . , 532 is also denoted as “ingoing edge” for the second node 520, . . . , 532 and as “outgoing edge” for the first node 520, . . . , 532.

In this embodiment, the nodes 520, . . . , 532 of the artificial neural network 500 can be arranged in layers 510, . . . , 513, wherein the layers can comprise an intrinsic order introduced by the edges 540, . . . , 542 between the nodes 520, . . . , 532. In particular, edges 540, . . . , 542 can exist only between neighboring layers of nodes. In the displayed embodiment, there is an input layer 510 comprising only nodes 520, . . . , 522 without an incoming edge, an output layer 513 comprising only nodes 531, 532 without outgoing edges, and hidden layers 511, 512 in-between the input layer 510 and the output layer 513. In general, the number of hidden layers 511, 512 can be chosen arbitrarily. The number of nodes 520, . . . , 522 within the input layer 510 usually relates to the number of input values of the neural network, and the number of nodes 531, 532 within the output layer 513 usually relates to the number of output values of the neural network.

In particular, a (real) number can be assigned as a value to every node 520, . . . , 532 of the neural network 500. Here, x⁽ⁿ⁾ᵢ denotes the value of the i-th node 520, . . . , 532 of the n-th layer 510, . . . , 513. The values of the nodes 520, . . . , 522 of the input layer 510 are equivalent to the input values of the neural network 500, the values of the nodes 531, 532 of the output layer 513 are equivalent to the output value of the neural network 500. Furthermore, each edge 540, . . . , 542 can comprise a weight being a real number, in particular, the weight is a real number within the interval [−1, 1] or within the interval [0, 1]. Here, w^{(m,n)}_{i,j} denotes the weight of the edge between the i-th node 520, . . . , 532 of the m-th layer 510, . . . , 513 and the j-th node 520, . . . , 532 of the n-th layer 510, . . . , 513. Furthermore, the abbreviation w⁽ⁿ⁾_{i,j} is defined for the weight w^{(n,n+1)}ᵢⱼ.

In particular, to calculate the output values of the neural network 500, the input values are propagated through the neural network. In particular, the values of the nodes 520, . . . , 532 of the (n+1)-th layer 510, . . . , 513 can be calculated based on the values of the nodes 520, . . . , 532 of the n-th layer 510, . . . , 513 by

$${x}^{{( n + 1 )}_{j}} = f ( {\sum }_{i} {x}^{{( n )}_{i}} \cdot {w}^{{( n )}_{i , j}} ) .$$

Herein, the function f is a transfer function (another term is “activation function”). Known transfer functions are step functions, sigmoid function (e.g., the logistic function, the generalized logistic function, the hyperbolic tangent, the Arctangent function, the error function, the smoothstep function) or rectifier functions. The transfer function is mainly used for normalization purposes.

In particular, the values are propagated layer-wise through the neural network, wherein values of the input layer 510 are given by the input of the neural network 500, wherein values of the first hid-den layer 511 can be calculated based on the values of the input layer 510 of the neural network, wherein values of the second hidden layer 512 can be calculated based in the values of the first hidden layer 511, etc.

In order to set the values w^{(m,n)}_{i,j} for the edges, the neural network 500 has to be trained using training data. In particular, training data comprises training input data and training output data (denoted as tᵢ). For a training step, the neural network 500 is applied to the training input data to generate calculated output data. In particular, the training data and the calculated output data comprise a number of values, said number being equal with the number of nodes of the output layer.

In particular, a comparison between the calculated output data and the training data is used to recursively adapt the weights within the neural network 500 (backpropagation algorithm). In particular, the weights are changed according to

$${w}^{{′ ( n )}_{i , j}} = {w}^{{( n )}_{i , j}} - γ \cdot {δ}^{{( n )}_{j}} \cdot {x}^{{( n )}_{i}}$$

wherein γ is a learning rate, and the numbers δ⁽ⁿ⁾ⱼ can be recursively calculated as

$${δ}^{{( n )}_{j}} = ( {\sum }_{k} {δ}^{{( n + 1 )}_{k}} \cdot {w}^{{( n + 1 )}_{j , k}} ) \cdot {f}^{′} ( {\sum }_{i} {x}^{{( n )}_{i}} \cdot {w}^{{( n )}_{i , j}} )$$

based on δ⁽ⁿ⁺¹⁾ⱼ, if the (n+1)-th layer is not the output layer, and

$${δ}^{{( n )}_{j}} = ( {x}^{{( n + 1 )}_{j}} - {t}^{{( n + 1 )}_{j}} ) \cdot {f}^{′} ( {x}^{{( n )}_{i}} \cdot {w}^{{( n )}_{i , j}} )$$

if the (n+1)-th layer is the output layer 513, wherein f′ is the first derivative of the activation function, and t⁽ⁿ⁺¹⁾ⱼ is the comparison training value for the j-th node of the output layer 513.

A convolutional neural network is a neural network that uses a convolution operation instead general matrix multiplication in at least one of its layers (so-called “convolutional layer”). In particular, a convolutional layer performs a dot product of one or more convolution kernels with the convolutional layer's input data/image, wherein the entries of the one or more convolution kernel are the parameters or weights that are adapted by training. In particular, one can use the Frobenius inner product and the ReLU activation function. A convolutional neural network can comprise additional layers, e.g., pooling layers, fully connected layers, and normalization layers.

By using convolutional neural networks input images can be processed in a very efficient way, because a convolution operation based on different kernels can extract various image features, so that by adapting the weights of the convolution kernel the relevant image features can be found during training. Furthermore, based on the weight-sharing in the convolutional kernels less parameters need to be trained, which prevents overfitting in the training phase and allows to have faster training or more layers in the network, improving the performance of the network.

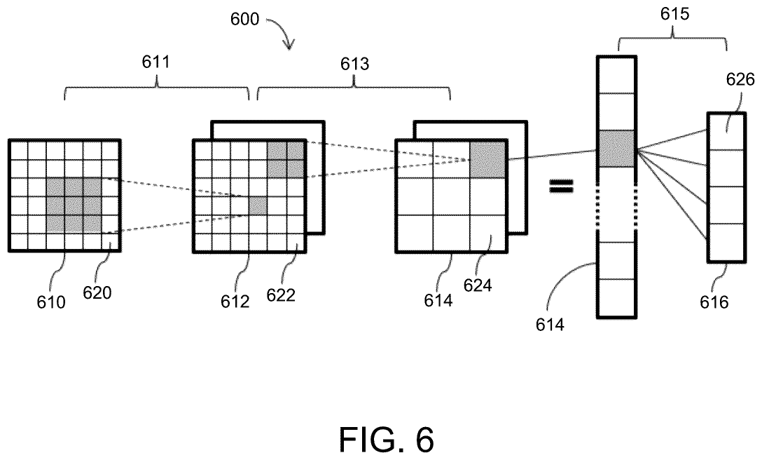

FIG. 6 shows an embodiment of a convolutional neural network 600 that may be used to implement one or more machine learning models described herein. In the displayed embodiment, the convolutional neural network comprises 600 an input node layer 610, a convolutional layer 611, a pooling layer 613, a fully connected layer 614 and an output node layer 616, as well as hidden node layers 612, 614. Alternatively, the convolutional neural network 600 can comprise several convolutional layers 611, several pooling layers 613 and several fully connected layers 615, as well as other types of layers. The order of the layers can be chosen arbitrarily, usually fully connected layers 615 are used as the last layers before the output layer 616.

In particular, within a convolutional neural network 600 nodes 620, 622, 624 of a node layer 610, 612, 614 can be considered to be arranged as a d-dimensional matrix or as a d-dimensional image. In particular, in the two-dimensional case the value of the node 620, 622, 624 indexed with i and j in the n-th node layer 610, 612, 614 can be denoted as x(n)[i, j]. However, the arrangement of the nodes 620, 622, 624 of one node layer 610, 612, 614 does not have an effect on the calculations executed within the convolutional neural network 600 as such, since these are given solely by the structure and the weights of the edges.

A convolutional layer 611 is a connection layer between an anterior node layer 610 (with node values x(n−1)) and a posterior node layer 612 (with node values x(n)). In particular, a convolutional layer 611 is characterized by the structure and the weights of the incoming edges forming a convolution operation based on a certain number of kernels. In particular, the structure and the weights of the edges of the convolutional layer 611 are chosen such that the values x(n) of the nodes 622 of the posterior node layer 612 are calculated as a convolution x(n)=K*x(n−1) based on the values x(n−1) of the nodes 620 anterior node layer 610, where the convolution * is defined in the two-dimensional case as

$${x}_{k}^{( n )} [ i , j ] = ( K * {x}^{( n - 1 )} ) [ i , j ] = {\sum }_{{i}^{′}} {\sum }_{{j}^{′}} K [ {i}^{′} , {j}^{′} ] \cdot {x}^{( n - 1 )} [ i - {i}^{′} , j - {j}^{′} ] .$$

Here the kernel K is a d-dimensional matrix (in this embodiment, a two-dimensional matrix), which is usually small compared to the number of nodes 620, 622 (e.g., a 3×3 matrix, or a 5×5 matrix). In particular, this implies that the weights of the edges in the convolution layer 611 are not independent, but chosen such that they produce said convolution equation. In particular, for a kernel being a 3×3 matrix, there are only 9 independent weights (each entry of the kernel matrix corresponding to one independent weight), irrespectively of the number of nodes 620, 622 in the anterior node layer 610 and the posterior node layer 612.

In general, convolutional neural networks 600 use node layers 610, 612, 614 with a plurality of channels, in particular, due to the use of a plurality of kernels in convolutional layers 611. In those cases, the node layers can be considered as (d+1)-dimensional matrices (the first dimension indexing the channels). The action of a convolutional layer 611 is then a two-dimensional example defined as

$${x}^{{( n )}_{b}} [ i , j ] = {\sum }_{a} {K}_{a , b} * {x}^{{( n - 1 )}_{a}} [ i , j ] = {\sum }_{a} {\sum }_{{i}^{′}} {\sum }_{{j}^{′}} {K}_{a , b} [ {i}^{′} , {j}^{′} ] \cdot {x}^{{( n - 1 )}_{a}} [ i - {i}^{′} , j - {j}^{′} ]$$

where x⁽ⁿ⁻¹⁾ₐ corresponds to the a-th channel of the anterior node layer 610, x⁽ⁿ⁾_{b} corresponds to the b-th channel of the posterior node layer 612 and K_{a,b} corresponds to one of the kernels. If a convolutional layer 611 acts on an anterior node layer 610 with A channels and outputs a posterior node layer 612 with B channels, there are A·B independent d-dimensional kernels K_{a,b}.

In general, in convolutional neural networks 600 activation functions are used. In this embodiment re ReLU (acronym for “Rectified Linear Units”) is used, with R(z)=max(0, z), so that the action of the convolutional layer 611 in the two-dimensional example is

$${x}^{{( n )}_{b}} [ i , j ] = R ( {\sum }_{a} ( {K}_{a , b} * {x}^{{( n - 1 )}_{a}} ) [ i , j ] ) = R ( {\sum }_{a} {\sum }_{{i}^{′}} {\sum }_{{j}^{′}} {K}_{a , b} [ {i}^{′} , {j}^{′} ] \cdot {x}^{{( n - 1 )}_{a}} [ i - {i}^{′} , j - {j}^{′} ] )$$

It is also possible to use other activation functions, e.g., ELU (acronym for “Exponential Linear Unit”), LeakyReLU, Sigmoid, Tanh or Softmax.

In the displayed embodiment, the input layer 610 comprises 36 nodes 620, arranged as a two-dimensional 6x6 matrix. The first hidden node layer 612 comprises 72 nodes 622, arranged as two two-dimensional 6×6 matrices, each of the two matrices being the result of a convolution of the values of the input layer with a 3×3 kernel within the convolutional layer 611. Equivalently, the nodes 622 of the first hidden node layer 612 can be interpreted as arranged as a three-dimensional 2×6×6 matrix, wherein the first dimension correspond to the channel dimension.

The advantage of using convolutional layers 611 is that spatially local correlation of the input data can exploited by enforcing a local connectivity pattern between nodes of adjacent layers, in particular by each node being connected to only a small region of the nodes of the preceding layer.

A pooling layer 613 is a connection layer between an anterior node layer 612 (with node values x(n−1)) and a posterior node layer 614 (with node values x(n)). In particular, a pooling layer 613 can be characterized by the structure and the weights of the edges and the activation function forming a pooling operation based on a non-linear pooling function f. For example, in the two-dimensional case the values x(n) of the nodes 624 of the posterior node layer 614 can be calculated based on the values x(n−1) of the nodes 622 of the anterior node layer 612 as

x⁽ⁿ⁾_{b}[i,j]=f(x⁽ⁿ⁻¹⁾[id₁, jd₂], . . . , x⁽ⁿ⁻¹⁾_{b}[(i+1)d₁−1, (j+1)d₂−1])

In other words, by using a pooling layer 613 the number of nodes 622, 624 can be reduced, by re-placing a number d1·d2 of neighboring nodes 622 in the anterior node layer 612 with a single node 622 in the posterior node layer 614 being calculated as a function of the values of said number of neighboring nodes. In particular, the pooling function f can be the max-function, the average or the L2-Norm. In particular, for a pooling layer 613 the weights of the incoming edges are fixed and are not modified by training.

The advantage of using a pooling layer 613 is that the number of nodes 622, 624 and the number of parameters is reduced. This leads to the amount of computation in the network being reduced and to a control of overfitting.

In the displayed embodiment, the pooling layer 613 is a max-pooling layer, replacing four neighboring nodes with only one node, the value being the maximum of the values of the four neighboring nodes. The max-pooling is applied to each d-dimensional matrix of the previous layer; in this embodiment, the max-pooling is applied to each of the two two-dimensional matrices, reducing the number of nodes from 72 to 18.

In general, the last layers of a convolutional neural network 600 are fully connected layers 615. A fully connected layer 615 is a connection layer between an anterior node layer 614 and a posterior node layer 616. A fully connected layer 613 can be characterized by the fact that a majority, in particular, all edges between nodes 614 of the anterior node layer 614 and the nodes 616 of the posterior node layer are present, and wherein the weight of each of these edges can be adjusted individually.

In this embodiment, the nodes 624 of the anterior node layer 614 of the fully connected layer 615 are displayed both as two-dimensional matrices, and additionally as non-related nodes (indicated as a line of nodes, wherein the number of nodes was reduced for a better presentability). This operation is also denoted as “flattening”. In this embodiment, the number of nodes 626 in the posterior node layer 616 of the fully connected layer 615 smaller than the number of nodes 624 in the anterior node layer 614. Alternatively, the number of nodes 626 can be equal or larger.

Furthermore, in this embodiment the Softmax activation function is used within the fully connected layer 615. By applying the Softmax function, the sum the values of all nodes 626 of the output layer 616 is 1, and all values of all nodes 626 of the output layer 616 are real numbers between 0 and 1. In particular, if using the convolutional neural network 600 for categorizing input data, the values of the output layer 616 can be interpreted as the probability of the input data falling into one of the different categories.

In particular, convolutional neural networks 600 can be trained based on the backpropagation algorithm. For preventing overfitting, methods of regularization can be used, e.g., dropout of nodes 620, . . . , 624, stochastic pooling, use of artificial data, weight decay based on the L1 or the L2 norm, or max norm constraints.

According to an aspect, the machine learning model may comprise one or more residual networks (ResNet). In particular, a ResNet is an artificial neural network comprising at least one jump or skip connection used to jump over at least one layer of the artificial neural network. In particular, a ResNet may be a convolutional neural network comprising one or more skip connections respectively skipping one or more convolutional layers. According to some examples, the ResNets may be represented as m-layer ResNets, where m is the number of layers in the corresponding architecture and, according to some examples, may take values of 34, 50, 101, or 152. According to some examples, such an m-layer ResNet may respectively comprise (m−2)/2 skip connections.

A skip connection may be seen as a bypass which directly feeds the output of one preceding layer over one or more bypassed layers to a layer succeeding the one or more bypassed layers. Instead of having to directly fit a desired mapping, the bypassed layers would then have to fit a residual mapping “balancing” the directly fed output.

Fitting the residual mapping is computationally easier to optimize than the directed mapping. What is more, this alleviates the problem of vanishing/exploding gradients during optimization upon training the machine learning models: if a bypassed layer runs into such problems, its contribution may be skipped by regularization of the directly fed output. Using ResNets thus brings about the advantage that much deeper networks may be trained.

In particular, a recurrent machine learning model is a machine learning model whose output does not only depend on the input value and the parameters of the machine learning model adapted by the training process, but also on a hidden state vector, wherein the hidden state vector is based on previous inputs used on for the recurrent machine learning model. In particular, the recurrent machine learning model can comprise additional storage states or additional structures that incorporate time delays or comprise feedback loops.

In particular, the underlying structure of a recurrent machine learning model can be a neural network, which can be denoted as recurrent neural network. Such a recurrent neural network can be described as an artificial neural network where connections between nodes form a directed graph along a temporal sequence. In particular, a recurrent neural network can be interpreted as directed acyclic graph. In particular, the recurrent neural network can be a finite impulse recurrent neural network or an infinite impulse recurrent neural network (wherein a finite impulse network can be unrolled and replaced with a strictly feedforward neural network, and an infinite impulse network cannot be unrolled and replaced with a strictly feedforward neural network).

In particular, training a recurrent neural network can be based on the BPTT algorithm (acronym for “backpropagation through time”), on the RTRL algorithm (acronym for “real-time recurrent learning”) and/or on genetic algorithms.

By using a recurrent machine learning model input data comprising sequences of variable length can be used. In particular, this implies that the method cannot be used only for a fixed number of input datasets (and needs to be trained differently for every other number of input datasets used as input), but can be used for an arbitrary number of input datasets. This implies that the whole set of training data, independent of the number of input datasets contained in different sequences, can be used within the training, and that training data is not reduced to training data corresponding to a certain number of successive input datasets.

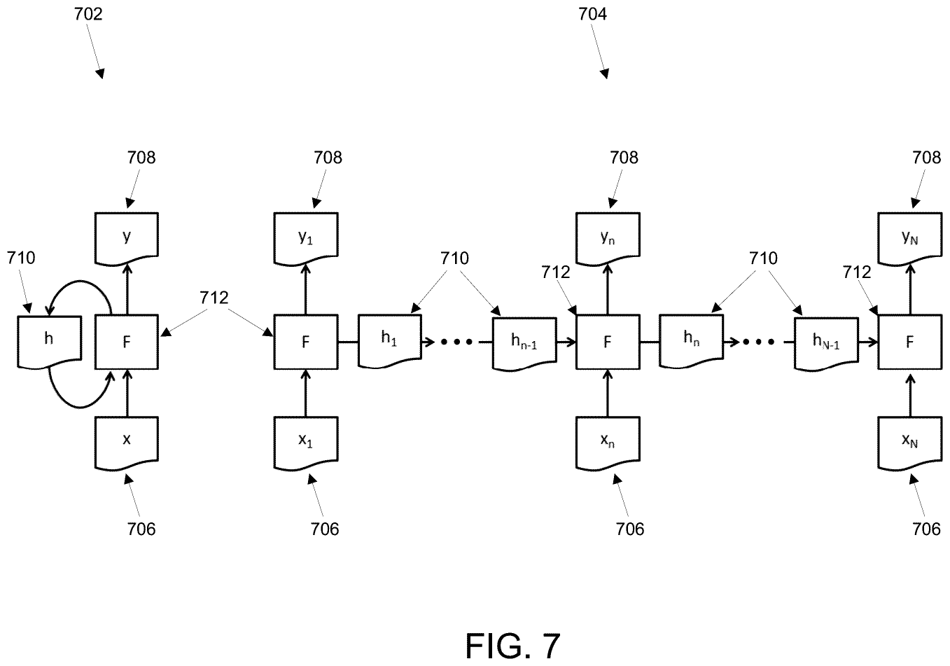

FIG. 7 shows the schematic structure of a recurrent machine learning model F, both in a recurrent representation 702 and in an unfolded representation 704, that may be used to implement one or more machine learning models described herein. The recurrent machine learning model takes as input several input datasets x, x₁, . . . , x_{N} 706 and creates a corresponding set of output datasets y, y₁, . . . , y_{N} 708. Furthermore, the output depends on a so-called hidden vector h, h₁, . . . , h_{N} 710, which implicitly comprises information about input datasets previously used as input for the recurrent machine learning model F 712. By using these hidden vectors h, h₁, . . . , h_{N} 710, a sequentiality of the input datasets can be leveraged.

In a single step of the processing, the recurrent machine learning model F 712 takes as input the hidden vector hₙ₋₁ created within the previous step and an input dataset xₙ. Within this step, the recurrent machine learning model F generates as output an updated hidden vector hn and an output dataset yₙ. In other words, one step of processing calculates (yₙ, hₙ)=F(xₙ, hₙ₋₁), or by splitting the recurrent machine learning model F 712 into a part F(y) calculating the output data and F(h) calculating the hidden vector, one step of processing calculates yₙ=F^{(y)}(xₙ, hₙ₋₁) and hₙ=F^{(h)}(xₙ, hₙ₋₁). For the first processing step, h₀ can be chosen randomly or filled with all entries being zero. The parameters of the recurrent machine learning model F 712 that were trained based on training datasets before do not change between the different processing steps.

In particular, the output data and the hidden vector of a processing step depend on all the previous input datasets used in the previous steps. yₙ=F^{(y)}(xₙ, F^{(h)}(xₙ₋₁, hₙ₋₂)) and hₙ=F^{(h)}(xₙ, F^{(h)}(xₙ₋₁, hₙ₋₂)).

Systems, apparatuses, and methods described herein may be implemented using digital circuitry, or using one or more computers using well-known computer processors, memory units, storage devices, computer software, and other components. Typically, a computer includes a processor for executing instructions and one or more memories for storing instructions and data. A computer may also include, or be coupled to, one or more mass storage devices, such as one or more magnetic disks, internal hard disks and removable disks, magneto-optical disks, optical disks, etc.

Systems, apparatuses, and methods described herein may be implemented using computers operating in a client-server relationship. Typically, in such a system, the client computers are located remotely from the server computer and interact via a network. The client-server relationship may be defined and controlled by computer programs running on the respective client and server computers.

Systems, apparatuses, and methods described herein may be implemented within a network-based cloud computing system. In such a network-based cloud computing system, a server or another processor that is connected to a network communicates with one or more client computers via a network. A client computer may communicate with the server via a network browser application residing and operating on the client computer, for example. A client computer may store data on the server and access the data via the network. A client computer may transmit requests for data, or requests for online services, to the server via the network. The server may perform requested services and provide data to the client computer(s). The server may also transmit data adapted to cause a client computer to perform a specified function, e.g., to perform a calculation, to display specified data on a screen, etc. For example, the server may transmit a request adapted to cause a client computer to perform one or more of the steps or functions of the methods and workflows described herein, including one or more of the steps or functions of FIG. 1 or 2. Certain steps or functions of the methods and workflows described herein, including one or more of the steps or functions of FIG. 1 or 2, may be performed by a server or by another processor in a network-based cloud-computing system. Certain steps or functions of the methods and workflows described herein, including one or more of the steps of FIG. 1 or 2, may be performed by a client computer in a network-based cloud computing system. The steps or functions of the methods and workflows described herein, including one or more of the steps of FIG. 1 or 2, may be performed by a server and/or by a client computer in a network-based cloud computing system, in any combination.

Systems, apparatuses, and methods described herein may be implemented using a computer program product tangibly embodied in an information carrier, e.g., in a non-transitory machine-readable storage device, for execution by a programmable processor; and the method and workflow steps described herein, including one or more of the steps or functions of FIG. 1 or 2, may be implemented using one or more computer programs that are executable by such a processor. A computer program is a set of computer program instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

A high-level block diagram of an example computer 802 that may be used to implement systems, apparatuses, and methods described herein is depicted in FIG. 8. Computer 802 includes a processor 804 operatively coupled to a data storage device 812 and a memory 810. Processor 804 controls the overall operation of computer 802 by executing computer program instructions that define such operations. The computer program instructions may be stored in data storage device 812, or other computer readable medium, and loaded into memory 810 when execution of the computer program instructions is desired. Thus, the method and workflow steps or functions of FIG. 1 or 2 can be defined by the computer program instructions stored in memory 810 and/or data storage device 812 and controlled by processor 804 executing the computer program instructions. For example, the computer program instructions can be implemented as computer executable code programmed by one skilled in the art to perform the method and workflow steps or functions of FIG. 1 or 2. Accordingly, by executing the computer program instructions, the processor 804 executes the method and workflow steps or functions of FIG. 1 or 2. Computer 802 may also include one or more network interfaces 806 for communicating with other devices via a network. Computer 802 may also include one or more input/output devices 808 that enable user interaction with computer 802 (e.g., display, keyboard, mouse, speakers, buttons, etc.).

Processor 804 may include both general and special purpose microprocessors, and may be the sole processor or one of multiple processors of computer 802. Processor 804 may include one or more central processing units (CPUs), for example. Processor 804, data storage device 812, and/or memory 810 may include, be supplemented by, or incorporated in, one or more application-specific integrated circuits (ASICs) and/or one or more field programmable gate arrays (FPGAs).

Data storage device 812 and memory 810 each include a tangible non-transitory computer readable storage medium. Data storage device 812, and memory 810, may each include high-speed random access memory, such as dynamic random access memory (DRAM), static random access memory (SRAM), double data rate synchronous dynamic random access memory (DDR RAM), or other random access solid state memory devices, and may include non-volatile memory, such as one or more magnetic disk storage devices such as internal hard disks and removable disks, magneto-optical disk storage devices, optical disk storage devices, flash memory devices, semiconductor memory devices, such as erasable programmable read-only memory (EPROM), electrically erasable programmable read-only memory (EEPROM), compact disc read-only memory (CD-ROM), digital versatile disc read-only memory (DVD-ROM) disks, or other non-volatile solid state storage devices.

Input/output devices 808 may include peripherals, such as a printer, scanner, display screen, etc. For example, input/output devices 808 may include a display device such as a cathode ray tube (CRT) or liquid crystal display (LCD) monitor for displaying information to the user, a keyboard, and a pointing device such as a mouse or a trackball by which the user can provide input to computer 802.

An image acquisition device 814 can be connected to the computer 802 to input image data (e.g., medical images) to the computer 802. It is possible to implement the image acquisition device 814 and the computer 802 as one device. It is also possible that the image acquisition device 814 and the computer 802 communicate wirelessly through a network. In a possible embodiment, the computer 802 can be located remotely with respect to the image acquisition device 814.

Any or all of the systems, apparatuses, and methods discussed herein may be implemented using one or more computers such as computer 802.

One skilled in the art will recognize that an implementation of an actual computer or computer system may have other structures and may contain other components as well, and that FIG. 8 is a high level representation of some of the components of such a computer for illustrative purposes.

Independent of the grammatical term usage, individuals with male, female or other gender identities are included within the term.

The foregoing Detailed Description is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the principles of the present invention and that various modifications may be implemented by those skilled in the art without departing from the scope and spirit of the invention. Those skilled in the art could implement various other feature combinations without departing from the scope and spirit of the invention.

The following is a list of non-limiting illustrative embodiments disclosed herein:

Illustrative embodiment 1. A computer-implemented method comprising: receiving input medical data associated with a medical domain; performing a clinical task based on the input medical data using a trained language model; and outputting results of the clinical task, wherein the trained language model is trained by: receiving domain-specific training data associated with the medical domain, and training a pretrained, instruction-tuned language model for the medical domain using the domain-specific training data.

Illustrative embodiment 2. The computer-implemented method according to illustrative embodiment 1, wherein the pretrained, instruction-tuned language model is trained by: performing general pretraining of a language model using non-domain-specific training data; and performing instruction tuning on the general pretrained language model using labeled training data.

Illustrative embodiment 3. The computer-implemented method of according to illustrative embodiment 2, wherein a same loss function is used for performing the general pretraining, performing the instruction tuning, and the training.

Illustrative embodiment 4. The computer-implemented method according to any one of illustrative embodiments 1-3, wherein training a pretrained, instruction-tuned language model for the medical domain using the domain-specific training data comprises: updating only parameters of certain layers of the pretrained, instruction-tuned language model at each iteration.

Illustrative embodiment 5. The computer-implemented method according to any one of illustrative embodiments 1-4, wherein training a pretrained, instruction-tuned language model for the medical domain using the domain-specific training data comprises: adding domain-specific vocabulary for the medical domain to the pretrained, instruction-tuned language model.

Illustrative embodiment 6. The computer-implemented method according to any one of illustrative embodiments 1-5, wherein the input medical data comprises a findings section of a radiology report and the clinical task comprises generation of an impressions section of the radiology report.

Illustrative embodiment 7. The computer-implemented method according to any one of illustrative embodiments 1-6, wherein the medical domain is radiology.

Illustrative embodiment 8. The computer-implemented method according to any one of illustrative embodiments 1-7, wherein the trained language model is a trained large language model.

Illustrative embodiment 9. An apparatus comprising: receiving input medical data associated with a medical domain; performing a clinical task based on the input medical data using a trained language model; and outputting results of the clinical task, wherein the trained language model is trained by: receiving domain-specific training data associated with the medical domain, and training a pretrained, instruction-tuned language model for the medical domain using the domain-specific training data.

Illustrative embodiment 10. The apparatus according to illustrative embodiment 9, wherein the pretrained, instruction-tuned language model is trained by: performing general pretraining of a language model using non-domain-specific training data; and performing instruction tuning on the general pretrained language model using labeled training data.

Illustrative embodiment 11. The apparatus according to illustrative embodiment 10, wherein a same loss function is used for performing the general pretraining, performing the instruction tuning, and the training.

Illustrative embodiment 12. The apparatus according to any one of illustrative embodiments 9-11, wherein training a pretrained, instruction-tuned language model for the medical domain using the domain-specific training data comprises: updating only parameters of certain layers of the pretrained, instruction-tuned language model at each iteration.

Illustrative embodiment 13. The apparatus according to any one of illustrative embodiments 9-12, wherein training a pretrained, instruction-tuned language model for the medical domain using the domain-specific training data comprises: adding domain-specific vocabulary for the medical domain to the pretrained, instruction-tuned language model.

Illustrative embodiment 14. A non-transitory computer-readable storage medium comprising instructions which, when executed by a computer, cause the computer to carry out operations comprising: receiving input medical data associated with a medical domain; performing a clinical task based on the input medical data using a trained language model; and outputting results of the clinical task, wherein the trained language model is trained by: receiving domain-specific training data associated with the medical domain, and training a pretrained, instruction-tuned language model for the medical domain using the domain-specific training data.

Illustrative embodiment 15. The non-transitory computer-readable storage medium according to illustrative embodiment 14, wherein the input medical data comprises a findings section of a radiology report and the clinical task comprises generation of an impressions section of the radiology report.

Illustrative embodiment 16. The non-transitory computer-readable storage medium according to any one of illustrative embodiments 14-15, wherein the medical domain is radiology.

Illustrative embodiment 17. The non-transitory computer-readable storage medium according to any one of illustrative embodiments 14-16, wherein the trained language model is a trained large language model.

Illustrative embodiment 18. A computer-implemented method comprising: receiving domain-specific training data associated with a medical domain; training a pretrained, instruction-tuned language model for the medical domain using the domain-specific training data; and outputting the trained language model.

Illustrative embodiment 19. The computer-implemented method according to illustrative embodiment 18, wherein the pretrained, instruction-tuned language model is trained by: performing general pretraining of a language model using non-domain-specific training data; and performing instruction tuning on the general pretrained language model using labeled training data.

Illustrative embodiment 20. The computer-implemented method according to any one of illustrative embodiments 18-19, wherein a same loss function is used for performing the general pretraining, performing the instruction tuning, and the training.

1. A computer-implemented method comprising:
receiving input medical data associated with a medical domain;
performing a clinical task based on the input medical data using a trained language model; and
outputting results of the clinical task,
wherein the trained language model is trained by:
receiving domain-specific training data associated with the medical domain, and
training a pretrained, instruction-tuned language model for the medical domain using the domain-specific training data.

2. The computer-implemented method of claim 1, wherein the pretrained, instruction-tuned language model is trained by:
performing general pretraining of a language model using non-domain-specific training data; and
performing instruction tuning on the general pretrained language model using labeled training data.

3. The computer-implemented method of claim 2, wherein a same loss function is used for performing the general pretraining, performing the instruction tuning, and the training.

4. The computer-implemented method of claim 1, wherein training a pretrained, instruction-tuned language model for the medical domain using the domain-specific training data comprises:
updating only parameters of certain layers of the pretrained, instruction-tuned language model at each iteration.

5. The computer-implemented method of claim 1, wherein training a pretrained, instruction-tuned language model for the medical domain using the domain-specific training data comprises:
adding domain-specific vocabulary for the medical domain to the pretrained, instruction-tuned language model.

6. The computer-implemented method of claim 1, wherein the input medical data comprises a findings section of a radiology report and the clinical task comprises generation of an impressions section of the radiology report.

7. The computer-implemented method of claim 1, wherein the medical domain is radiology.

8. The computer-implemented method of claim 1, wherein the trained language model is a trained large language model.

9. An apparatus comprising:
receiving input medical data associated with a medical domain;
performing a clinical task based on the input medical data using a trained language model; and
outputting results of the clinical task,
wherein the trained language model is trained by:
receiving domain-specific training data associated with the medical domain, and
training a pretrained, instruction-tuned language model for the medical domain using the domain-specific training data.

10. The apparatus of claim 9, wherein the pretrained, instruction-tuned language model is trained by:
performing general pretraining of a language model using non-domain-specific training data; and
performing instruction tuning on the general pretrained language model using labeled training data.

11. The apparatus of claim 10, wherein a same loss function is used for performing the general pretraining, performing the instruction tuning, and the training.

12. The apparatus of claim 9, wherein training a pretrained, instruction-tuned language model for the medical domain using the domain-specific training data comprises:
updating only parameters of certain layers of the pretrained, instruction-tuned language model at each iteration.

13. The apparatus of claim 9, wherein training a pretrained, instruction-tuned language model for the medical domain using the domain-specific training data comprises:
adding domain-specific vocabulary for the medical domain to the pretrained, instruction-tuned language model.

14. A non-transitory computer-readable storage medium comprising instructions which, when executed by a computer, cause the computer to carry out operations comprising:
receiving input medical data associated with a medical domain;
performing a clinical task based on the input medical data using a trained language model; and
outputting results of the clinical task,
wherein the trained language model is trained by:
receiving domain-specific training data associated with the medical domain, and
training a pretrained, instruction-tuned language model for the medical domain using the domain-specific training data.

15. The non-transitory computer-readable storage medium of claim 14, wherein the input medical data comprises a findings section of a radiology report and the clinical task comprises generation of an impressions section of the radiology report.

16. The non-transitory computer-readable storage medium of claim 14, wherein the medical domain is radiology.

17. The non-transitory computer-readable storage medium of claim 14, wherein the trained language model is a trained large language model.

18. A computer-implemented method comprising:
receiving domain-specific training data associated with a medical domain;
training a pretrained, instruction-tuned language model for the medical domain using the domain-specific training data; and
outputting the trained language model.

19. The computer-implemented method of claim 18, wherein the pretrained, instruction-tuned language model is trained by:
performing general pretraining of a language model using non-domain-specific training data; and
performing instruction tuning on the general pretrained language model using labeled training data.

20. The computer-implemented method of claim 19, wherein a same loss function is used for performing the general pretraining, performing the instruction tuning, and the training.
